(19) United States
(12) Patent Application Publication
(10) Pub. No.: US 2017/0202766 A1
(43) Pub. Date: Jul. 20, 2017

(54) USE OF SPICULISPORIC ACID AND/OR A SALT THEREOF AS ANTIDANDRUFF AGENT, AND COSMETIC TREATMENT PROCESS EMPLOYING SAME

(71) Applicant: L'OREAL, Paris (FR)

(72) Inventors: Caroline SIRICHANDRA, Joinville le Pont (FR); Sandie LESCH, Paris (FR)

(21) Appl. No.: 15/324,807

(22) PCT Filed: Jun. 26, 2015
(86) PCT No.: PCT/EP2015/064494

§ 371 Date: Jan. 9, 2017

(30) Foreign Application Priority Data
Jul. 10, 2014 (FR) 1456666

(51) Int. Cl. A61K 8/49; A61K 8/44; A61Q 5/00

(52) U.S. Cl. CPC A61K 8/4973; A61Q 5/006; A61K 8/44

(57) ABSTRACT

Use of spiculisporic acid and/or a salt thereof as antidandruff agent, and cosmetic treatment process employing same. The present invention relates to the cosmetic use, especially for hair, of spiculisporic acid and/or a salt thereof as anti dandruff agent. The invention also relates to a cosmetic treatment process intended for eliminating dandruff and/or for reducing the amount thereof, comprising the application, to the hair and/or the scalp, of spiculisporic acid and/or a salt thereof, or of a cosmetic composition comprising it/them.

The present invention relates to the cosmetic use, especially for hair, of spiculisporic acid and/or a salt thereof, as antidandruff agent, in particular in the cosmetic treatment of dandruff conditions associated with the excessive proliferation of yeasts of the Malassezia genus on the scalp.

Dandruff problems affect up to 50% of the worldwide population. They affect both men and women and are perceived as having a very negative psychosocial impact. The appearance of dandruff is disagreeable both aesthetically and because of the discomfort it causes (especially tingling or itching), and as such many people confronted with this problem wish to eliminate it efficiently and permanently. Dandruff corresponds to excessive and visible desquamation of the scalp resulting from excessively rapid multiplication of the epidermal cells and their abnormal maturation. This phenomenon may be caused especially by excessively aggressive hair treatments, extreme climatic conditions, stress, diet, fatigue and pollution. Dandruff conditions usually result from a disorder of the scalp microflora and more particularly from excessive colonization by a fungus which belongs to the family of yeasts of the Malassezia genus and which is naturally present on the scalp. Many antidandruff treatments have been developed with the principal objective of eradicating Malassezia yeasts from the scalp. Thus, the activity of the antidandruff active agents currently used, such as zinc pyrithione, piroctone olamine or selenium disulfide, is based mainly on their fungicidal property. However, these antidandruff agents are not completely satisfactory in terms of effectiveness (immediate effectiveness and/or duration of the effect) and/or in terms of their impact on the environment.

Thus, finding solutions to formulate environmentally friendly cosmetic products which are just as effective has become a major challenge in order to respond to the needs of consumers.

An aim of the present invention is to provide an antidandruff agent that is nonirritant to the skin and the scalp, and that is as effective as the known antidandruff agents, while having a more favourable environmental impact, especially fulfilling criteria of naturalness and/or renewability.

Another aim of the invention is to propose an active agent that can re-establish, and maintain at a normal level, the ecoflora of the scalp and especially prevent excessive colonization of the scalp by Malassezia sp.

The Applicant has discovered surprisingly that the cosmetic use of spiculisporic acid and/or a salt thereof enabled effective treatment of dandruff conditions, especially those associated with the proliferation of yeasts of the Malassezia genus. It was observed that, by using the compound according to the invention, the excessive number of yeasts of the Malassezia genus could be reduced and consequently the number of dandruff could be reduced.

Spiculisporic acid is a natural and environmentally friendly biosurfactant from a renewable source and is readily biodegradable.

A subject-matter of the present invention is therefore the cosmetic use of spiculisporic acid and/or a salt thereof as antidandruff agent.

Another subject-matter of the present invention is a cosmetic treatment process intended for eliminating dandruff and/or for reducing the amount thereof, comprising the application, to the hair and/or the scalp, of spiculisporic acid and/or a salt thereof, or of a cosmetic composition comprising it/them.

### Spiculisporic Acid

Spiculisporic acid, also known by the name 4,5-dicarboxy-4-pentadecanolide, has the following formula:

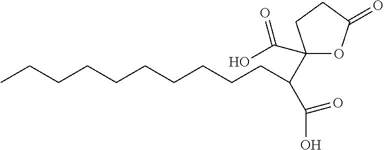 [SMILES: CCCCCCCCCCC(C(=O)O)C1(C(=O)O)CCC(=O)O1]

The spiculisporic acid which is usable in the context of the invention may be in salified or unsalified form.

When it is in the form of a salt, it may be an organic or inorganic salt. In this case, the spiculisporic acid may be used in a mixture with an organic or inorganic base. This base is a neutralizing base, that is to say it enables the spiculisporic acid to be neutralized so as to form a salt of said acid. In particular, the bases may be selected from:

aqueous ammonia,

alkanolamines, such as mono-, di- and triethanolamines, isopropanolamine and 2-amino-2-methyl-1-propanol,

oxyethylenated and/or oxypropylenated ethylenediamines,

inorganic or organic hydroxides.

The bases are preferably selected from hydroxides of an alkali metal, hydroxides of an alkaline-earth metal, for instance sodium hydroxide or potassium hydroxide, hydroxides of a transition metal, such as hydroxides of metals from Groups III, IV, V and VI of the Periodic Table of the Elements, hydroxides of lanthanides or actinides, quaternary ammonium hydroxides and guanidinium hydroxide. The hydroxide may be formed in situ, such as for instance guanidine hydroxide, formed by reacting calcium hydroxide and guanidine carbonate.

alkali metal silicates, such as sodium metasilicates,

amino acids, preferably basic amino acids, such as arginine, lysine, ornithine, citrulline and histidine,

carbonates and bicarbonates, particularly of a primary amine, secondary amine or tertiary amine, of an alkali metal or alkaline-earth metal, or of ammonium, and

the compounds of following formula:

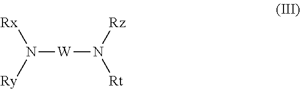

in which W is a C₁-C₆ alkylene residue optionally substituted with a hydroxyl group or a C₁-C₆ alkyl group; Rx, Ry, Rz and Rt, which may be identical or different, represent a hydrogen atom or a C₁-C₆ alkyl, C₁-C₆ hydroxyalkyl or C₁-C₆ aminoalkyl group. Mention may especially be made of 1,3-diaminopropane, 1,3-diamino-2propanol, spermine and spermidine.

Most particularly, the base may be selected from inorganic hydroxides, such as sodium hydroxide, potassium hydroxide, lithium hydroxide, caesium hydroxide, calcium hydroxide, magnesium hydroxide, zinc hydroxide; basic amino acids, such as lysine, arginine, histidine, citrulline, ornithine; alkanolamines, such as monoethanolamine, diethanolamine, 2-(dimethylamino)ethanol, triethanolamine, triisopropanolamine, diisopropanolamine, monoisopropanolamine; aqueous ammonia; guanidine carbonate; and mixtures thereof.

Preferably, the base is selected from arginine, triethanolamine, potassium hydroxide, sodium hydroxide and mixtures thereof.

Thus, it is most particularly preferred to use the sodium, potassium, triethanolamine and/or arginine salts of spiculisporic acid.

Preferably, the weight ratio spiculisporic acid/base is between 1/0.5 and 1/1.1, preferably from 1/0.6 to 1/0.8, and better still from 1/0.6 to 1/0.7.

### Cosmetic Composition

The spiculisporic acid may be carried in a cosmetic composition so as to be applied to the hair and/or the scalp.

Preferably, said cosmetic composition comprises a cosmetically acceptable medium, that is to say a medium compatible with topical application to keratin materials, especially the scalp and the hair.

Preferably, the pH of said cosmetic composition is between 4 and 7.5, notably between 4.5 and 7, and in particular between 4.7 and 6.5.

Preferably, the spiculisporic acid and/or the salts thereof is present in the cosmetic composition in an amount of between 0.5 and 20% by weight, in particular between 1 and 15% by weight, more particularly between 1.5 and 10% by weight, relative to the total weight of the composition.

Preferably, said cosmetic composition is a composition for the hair, which may be a rinse-out or a leave-in composition. The composition is preferably in the form of a shampoo, a cream, a mousse (aerosol or non-aerosol), a paste, a gel, an emulsion, a lotion or even a stick. Preferably, the composition for the hair is a shampoo, a gel or a lotion.

The cosmetic composition preferably comprises water and/or one or more organic solvents that may be selected from linear or branched C₁-C₆ monoalcohols, such as ethanol, isopropanol, tert-butanol or n-butanol; polyols, such as glycerol, propylene glycol, hexylene glycol (or 2-methyl-2,4-pentanediol) and polyethylene glycols; polyol ethers, such as dipropylene glycol monomethyl ether; and mixtures thereof.

Preferably, the cosmetic composition comprises water in an amount ranging from 30 to 98% by weight, especially from 40 to 95% by weight, better from 50 to 90% by weight, relative to the total weight of the composition.

Preferably, the cosmetic composition comprises the organic solvent(s) in an amount ranging from 0.05 to 60%, preferably from 0.5 to 50% and better still from 1 to 40% by weight, relative to the total weight of the cosmetic composition.

Said cosmetic composition according to the invention may also comprise at least one customary cosmetic ingredient, especially selected from plant, mineral, animal or synthetic oils; liquid fatty alcohols; liquid fatty esters; solid fatty substances and notably waxes, solid fatty esters, solid alcohols; anionic, cationic, amphoteric and nonionic surfactants; anionic, nonionic, amphoteric and cationic polymers; sunscreens; moisturizers; antidandruff agents other than spiculisporic acid; antioxidants; agents for combating hair loss; pearlizing and opacifying agents; plasticizers or coalescence agents; hydroxy acids; fillers; silicones and in particular polydimethylsiloxanes (PDMSs); perfumes; basifying agents or acidifying agents; aldehydes, DHA; polymeric or non-polymeric thickeners, and notably associative polymers; preservatives; sequestrants (EDTA and salts thereof); colorants. The composition can, of course, comprise several cosmetic ingredients appearing in the above list. A person skilled in the art will take care to select the ingredients making up the composition, and also the amounts thereof, such that the advantageous properties of the composition according to the invention are not, or are not substantially, adversely affected by the envisaged addition.

The application of said acid or a salt thereof or the composition comprising same to keratin materials, such as the hair and/or the scalp, can be followed, or not, by a rinsing step, for example with water.

The invention is illustrated in more detail in the following examples.

### EXAMPLE 1

### Demonstration of Activity on Malassezia Furfur

### Protocol:

The model strain used is: Malassezia furfur ATCC 12078 (yeast).

The strain is placed in contact with the formula to be tested in a suitable liquid culture medium in the following ratios:
10% of the microbial inoculum at 10⁷ microorganisms/ml
10% of the formula to be tested
80% of liquid culture medium (sabouraud broth+olive oil).

In parallel, a growth control, in which the formula to be tested is replaced with diluent (tryptone salt), is prepared under the same conditions.

The samples are placed in a rotary incubator at 35° C. and stirred throughout the duration of the test.

After 2, 6 and 24 hours of contact, the number of revivable microorganisms remaining in the mixture is evaluated.

The results are expressed as a logarithm of the number of microorganisms per millilitre of mixture.

### Results:

1/

Formula A comprising (% by weight):

10% spiculisporic acid

5.84% L-arginine (R_{base}/R_{acid} ratio=1.1)

water q.s. 100%

Change in the number of revivable microorganisms per ml of sample (in log):

| | t0 | t2h | t6h | t24h |
|---|---|---|---|---|
| Formula A | 6.0 | 5.3 | 4.7 | 2.4 |
| Control | 6.0 | 5.6 | 5.8 | 5.8 |

It is observed that, relative to the growth control, formula A according to the invention has good antimicrobial activity on M. furfur (reduction by 3.4 log units at 24 hours).

2/

Formula B comprising (% by weight):

5% spiculisporic acid

2.92% L-arginine (R_{base}/R_{acid} ratio=1.1)

water q.s. 100%

Change in the number of revivable microorganisms per ml of sample (in log):

| | t0 | t2h | t6h | t24h |
|---|---|---|---|---|
| Formula B | 6.0 | 5.3 | 4.8 | 2.1 |
| Control | 6.0 | 5.6 | 5.8 | 5.8 |

It is observed that, relative to the growth control, formula B according to the invention has good antimicrobial activity on M. furfur (reduction by 3.7 log units at 24 hours).

### EXAMPLE 2

A hair lotion is prepared, comprising (% by weight):

| | |
|---|---|
| spiculisporic acid | 5% |
| L-arginine | 3.1% |
| water | qsp |
| | 100% |

This lotion can be applied to the hair and make it possible to combat dandruff.

### EXAMPLE 3

An antidandruff shampoo is prepared, comprising (% by weight):

| | |
|---|---|
| sodium lauryl sulfate | 10% |
| cocoylbetaine | 5% |
| cocamide MEA | 1% |
| spiculisporic acid | 2% |
| L-arginine | 1.25% |
| preservative | q.s. |
| water | qsp |
| | 100% |

1. Cosmetic use, especially for hair, of spiculisporic acid and/or a salt thereof as antidandruff agent.

2. Use according to claim 1, in which spiculisporic acid is used in a mixture with an organic or inorganic base, preferably selected from:
aqueous ammonia,
alkanolamines, such as mono-, di- and triethanolamines, isopropanolamine and 2-amino-2-methyl-1-propanol,
oxyethylenated and/or oxypropylenated ethylenediamines,
inorganic or organic hydroxides,
alkali metal silicates, such as sodium metasilicates,
amino acids, preferably basic amino acids, such as arginine, lysine, ornithine, citrulline and histidine,
carbonates and bicarbonates, particularly of a primary amine, secondary amine or tertiary amine, of an alkali metal or alkaline-earth metal, or of ammonium, and
the compounds of following formula: 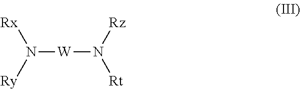
in which W is a C₁-C₆ alkylene residue optionally substituted with a hydroxyl group or a C₁-C₆ alkyl group; Rx, Ry, Rz and Rt, which may be identical or different, represent a hydrogen atom or a C₁-C₆ alkyl, C₁-C₆ hydroxyalkyl or C₁-C₆ aminoalkyl group; such as 1,3-diaminopropane, 1,3-diamino-2-propanol, spermine or spermidine;
preferably, the base is selected from inorganic hydroxides, such as sodium hydroxide, potassium hydroxide, lithium hydroxide, caesium hydroxide, calcium hydroxide, magnesium hydroxide, zinc hydroxide; basic amino acids, such as lysine, arginine, histidine, citrulline, ornithine; alkanolamines, such as monoethanolamine, diethanolamine, 2-(dimethylamino)ethanol, triethanolamine, triisopropanolamine, diisopropanolamine, monoisopropanolamine; aqueous ammonia; guanidine carbonate; and mixtures thereof;
and even better still the base is selected from arginine, triethanolamine, potassium hydroxide, sodium hydroxide and mixtures thereof.

3. Use according to either of the preceding claims, in which the spiculisporic acid and/or the salts thereof is carried in a cosmetic composition in which it is present in an amount of between 0.5 and 20% by weight, in particular between 1 and 15% by weight, more particularly between 1.5 and 10% by weight, relative to the total weight of the composition.

4. Use according to claim 3, in which said cosmetic composition comprises water and/or one or more organic solvents that may be selected from linear or branched C₁-C₆ monoalcohols, such as ethanol, isopropanol, tert-butanol or n-butanol; polyols, such as glycerol, propylene glycol, hexylene glycol (or 2-methyl-2,4pentanediol) and polyethylene glycols; polyol ethers, such as dipropylene glycol monomethyl ether; and mixtures thereof.

5. Use according to either of claims 3 and 4, in which said cosmetic composition comprises water in an amount ranging from 30 to 98% by weight, especially from 40 to 95% by weight, better from 50 to 90% by weight, relative to the total weight of the composition; and/or the cosmetic composition comprises the organic solvent(s) in an amount ranging from 0.05 to 60%, preferably from 0.5 to 50% and better still from 1 to 40% by weight, relative to the total weight of the cosmetic composition.

6. Use according to one of claims 3 to 5, in which said cosmetic composition comprises at least one customary cosmetic ingredient, especially selected from plant, mineral, animal or synthetic oils; liquid fatty alcohols; liquid fatty esters; solid fatty substances and notably waxes, solid fatty esters, solid alcohols; anionic, cationic, amphoteric and nonionic surfactants; anionic, nonionic, amphoteric and cationic polymers; sunscreens; moisturizers; antidandruff agents other than spiculisporic acid; antioxidants; agents for combating hair loss; pearlizing and opacifying agents; plasticizers or coalescence agents; hydroxy acids; fillers; silicones and in particular polydimethylsiloxanes (PDMSs); perfumes; basifying agents or acidifying agents; aldehydes, DHA; polymeric or non-polymeric thickeners, and notably associative polymers; preservatives; sequestrants (EDTA and salts thereof); colorants.

7. Use according to one of claims 3 to 6, in which the pH of said cosmetic composition is between 4 and 7.5, notably between 4.5 and 7, and in particular between 4.7 and 6.5.

8. Use according to one of claims 3 to 7, in which said cosmetic composition is a composition for the hair, which may be in the form of a shampoo, a cream, an aerosol or non-aerosol mousse, a paste, a gel, an emulsion, a lotion or a stick.

9. Cosmetic treatment process intended for eliminating dandruff and/or for reducing the amount thereof, comprising the application, to the hair and/or the scalp, of spiculisporic acid and/or a salt thereof, or of a cosmetic composition comprising it/them.

10. Process according to claim 9, in which spiculisporic acid is used in a mixture with an organic or inorganic base, preferably selected from:
aqueous ammonia,
alkanolamines, such as mono-, di- and triethanolamines, isopropanolamine and 2-amino-2-methyl-1-propanol,
oxyethylenated and/or oxypropylenated ethylenediamines,
inorganic or organic hydroxides,
alkali metal silicates, such as sodium metasilicates,
amino acids, preferably basic amino acids, such as arginine, lysine, ornithine, citrulline and histidine,
carbonates and bicarbonates, particularly of a primary amine, secondary amine or tertiary amine, of an alkali metal or alkaline-earth metal, or of ammonium, and
the compounds of following formula: 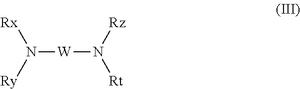
in which W is a C₁-C₆ alkylene residue optionally substituted with a hydroxyl group or a C₁-C₆ alkyl group; Rx, Ry, Rz and Rt, which may be identical or different, represent a hydrogen atom or a C₁-C₆ alkyl, C₁-C₆ hydroxyalkyl or C₁-C₆ aminoalkyl group; such as 1,3-diaminopropane, 1,3-diamino-2-propanol, spermine or spermidine;
preferably, the base is selected from inorganic hydroxides, such as sodium hydroxide, potassium hydroxide, lithium hydroxide, caesium hydroxide, calcium hydroxide, magnesium hydroxide, zinc hydroxide; basic amino acids, such as lysine, arginine, histidine, citrulline, ornithine; alkanolamines, such as monoethanolamine, diethanolamine, 2-(dimethylamino)ethanol, triethanolamine, triisopropanolamine, diisopropanolamine, monoisopropanolamine; aqueous ammonia; guanidine carbonate; and mixtures thereof;
and better still the base is selected from arginine, triethanolamine, potassium hydroxide, sodium hydroxide and mixtures thereof.

11. Process according to either of claims 9 and 10, in which the spiculisporic acid and/or the salts thereof is present in the cosmetic composition in an amount of between 0.5 and 20% by weight, in particular between 1 and 15% by weight, more particularly between 1.5 and 10% by weight, relative to the total weight of the composition.

12. Process according to one of claims 9 to 11, in which said cosmetic composition comprises water and/or one or more organic solvents that may be selected from linear or branched C₁-C₆ monoalcohols, such as ethanol, isopropanol, tertbutanol or n-butanol; polyols, such as glycerol, propylene glycol, hexylene glycol (or 2-methyl-2,4-pentanediol) and polyethylene glycols; polyol ethers, such as dipropylene glycol monomethyl ether; and mixtures thereof;
notably said cosmetic composition comprises water in an amount ranging from 30 to 98% by weight, especially from 40 to 95% by weight, better still from 50 to 90% by weight, relative to the total weight of the composition; and/or the cosmetic composition comprises the organic solvent(s) in an amount ranging from 0.05 to 60%, preferably from 0.5 to 50%, and better still from 1 to 40% by weight, relative to the total weight of the cosmetic composition.

13. Process according to one of claims 9 to 12, in which said cosmetic composition comprises at least one customary cosmetic ingredient, especially selected from plant, mineral, animal or synthetic oils; liquid fatty alcohols; liquid fatty esters; solid fatty substances and notably waxes, solid fatty esters, solid alcohols; anionic, cationic, amphoteric and nonionic surfactants; anionic, nonionic, amphoteric and cationic polymers; sunscreens; moisturizers; antidandruff agents other than spiculisporic acid; antioxidants; agents for combating hair loss; pearlizing and opacifying agents; plasticizers or coalescence agents; hydroxy acids; fillers; silicones and in particular polydimethylsiloxanes (PDMSs); perfumes; basifying agents or acidifying agents; aldehydes, DHA; polymeric or non-polymeric thickeners, and notably associative polymers; preservatives; sequestrants (EDTA and salts thereof); colorants.

14. Process according to one of claims 9 to 13, in which the pH of said cosmetic composition is between 4 and 7.5, notably between 4.5 and 7, and in particular between 4.7 and 6.5.

15. Process according to one of claims 9 to 14, in which said cosmetic composition is a composition for the hair, which may be in the form of a shampoo, a cream, an aerosol or non-aerosol mousse, a paste, a gel, an emulsion, a lotion or a stick.
